(19) United States
(12) Patent Application Publication
(10) Pub. No.: US 2009/0018182 A1
(43) Pub. Date: Jan. 15, 2009

(54) Crystalline forms of atorvastatin

(76) Inventors: Sigalit Levi, Modi'in (IL); Revital Lifshitz-Liron, Hertzlia (IL); Sharon Avhar-Maydan, Givataym (IL)

Correspondence Address: KENYON & KENYON LLP, ONE BROADWAY, NEW YORK, NY, 10004, US

(21) Appl. No.: 11/824,099

(22) Filed: Jun. 28, 2007

Related U.S. Application Data
(60) Provisional application No. 60/816,881, filed on Jun. 28, 2006.
(60) Provisional application No. 60/837,933, filed on Aug. 16, 2006.

(51) Int. Cl. A61K 31/40; C07D 207/34

(52) U.S. Cl. 514/423; 548/537

(57) ABSTRACT

Novel forms of atorvastatin hemi-calcium have been prepared and characterized. These novel forms are particularly useful in pharmaceutical compositions.

### RELATED APPLICATIONS

This application claims the benefit of Provisional Application Ser. No. 60/816,881, filed Jun. 28, 2006, and to Provisional Application Ser. No. 60/837,933, filed Aug. 16, 2006. The contents of those applications are incorporated herein in their entirety by reference.

### FIELD OF INVENTION

The present invention relates to crystalline forms of atorvastatin hemi-calcium, processes for their preparation and pharmaceutical compositions comprising the crystalline atorvastatin hemi-calcium forms.

### BACKGROUND OF THE INVENTION

Atorvastatin (ATV), ([R-(R*,R*)]-2-(4-fluorophenyl)-β,δ-dihydroxy-5-(1-methylethyl)-3 phenyl-4-[(phenylamino)carbonyl]-1H-pyrrole-1-heptanoic acid), depicted in lactone form in formula (I) and its calcium salt trihydrate of formula (II) (water molecules not shown) are described, inter alia, in U.S. Pat. Nos. 4,681,893 and 5,273,995, and in U.S. Provisional Application No. 60/166,153, filed Nov. 17, 2000, all of which are herein incorporated by reference.

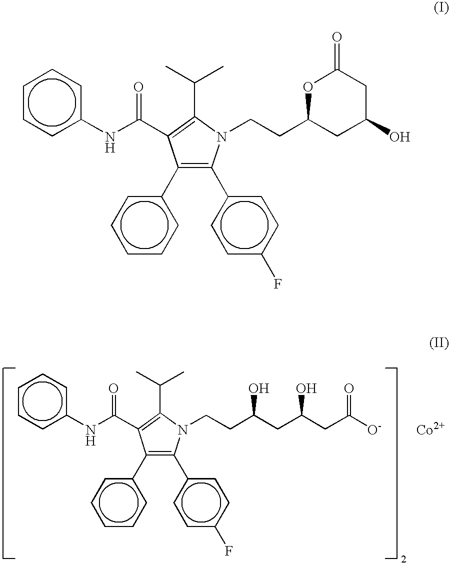

Atorvastatin is a member of the class of drugs called statins. Statin drugs are currently the most therapeutically effective drugs available for reducing low density lipoprotein (LDL) particle concentration in the blood stream of patients at risk for cardiovascular disease. A high level of LDL in the bloodstream has been linked to the formation of coronary lesions which obstruct the flow of blood and can rupture and promote thrombosis. Goodman and Gilman, The Pharmacological Basis of Therapeutics 879 (9th ed., 1996). Reducing plasma LDL levels has been shown to reduce the risk of clinical events in patients with cardiovascular disease and patients who are free of cardiovascular disease but who have hypercholesterolemia. Scandinavian Simvastatin Survival Study Group, 1994; Lipid Research Clinics Program, 1984a, 1984b.

Atorvastatin hemi-calcium salt trihydrate is marketed under the name LIPITOR® by Pfizer, Inc.

Processes for preparing atorvastatin and its hemi-calcium salt are disclosed in U.S. Patent Application Publication No. 2002/0099224; U.S. Pat. Nos. 5,273,995; 5,298,627; 5,003,080; 5,097,045; 5,124,482; 5,149,837; 5,216,174; 5,245,047; and 5,280,126; Baumann, K. L. et al. Tet. Lett. 1992, 33, 2283-2284, which are hereby incorporated by reference in their entirety and in particular for providing methods to prepare atorvastatin and atorvastatin hemi-calcium. Atorvastatin is also disclosed in U.S. Pat. No. 4,681,893.

The hemi-calcium salt depicted in formula (II) is disclosed in U.S. Pat. No. 5,273,995. The '995 patent states that the amorphous hemi-calcium salt is obtained by crystallization from a brine solution resulting from the transposition of the sodium salt with CaCl₂ and further purified by recrystallization from ethyl acetate and hexane.

The following crystalline forms of atorvastatin hemi-calcium: crystalline atorvastatin hydrate characterized by a powder X-ray diffraction pattern having peaks at 9.2, 9.5, 10.3, 10.6, 11.9, 12.2, 17.1, 19.5, 21.6, 22.0, 22.7, 23.3, 23.7, 24.4, 28.9, and 29.2 degrees two theta, denominated Form I; crystalline atorvastatin hydrate characterized by a powder X-ray diffraction pattern having peaks at 5.6, 7.4, 8.5, 9.0, 12.4 (broad), 15.8 (broad), 17.1-17.4 (broad), 19.5, 20.5, 22.7-23.2 (broad), 25.7 (broad), and 29.5 degrees two theta, denominated Form II; crystalline atorvastatin hydrate characterized by a powder X-ray diffraction pattern having peaks at 4.1, 5.0, 5.8, 7.7, 8.5, 16.0, 16.6, 17.7, 18.3, 18.9, 19.5, 20.0, 20.3, 21.1, 21.7, 23.3, 24.4, 25.0, and 25.4 degrees two theta, denominated Form III; and crystalline atorvastatin hydrate characterized by a powder X-ray diffraction pattern having peaks at 4.9, 5.4, 5.9, 8.0, 9.7, 10.4, 12.4, 17.7, 18.4, 19.2, 19.6, 21.7, 23.0, 23.7, and 24.1 degrees two theta, denominated Form IV, are the subjects of U.S. Pat. Nos. 5,959,156 and 6,121,461, assigned to Warner-Lambert. Crystalline atorvastatin hemi-calcium characterized by X-ray powder diffraction having peaks at about 5.3 and 8.3 degrees two theta and a broad peak at about 18-23 degrees two theta, denominated Form V, is disclosed in commonly-owned International Publication No. WO 01/36384. Form V is also said to have solid state ¹³C NMR signals at about 21.9, 25.9, 118.9, 122.5, 128.7, 161.0 and 167.1 ppm. Other crystalline forms of atorvastatin hemi-calcium are also disclosed in International Publication Nos. WO 02/43732, WO 02/41834, and WO 03/070702. One of these crystalline forms is denominated Form VIII, and is characterized by the powder X-ray diffraction pattern having peaks at 6.9, 9.3, 9.6, 16.3, 17.1, 19.2, 20.0, 21.6, 22.4, 23.9, 24.7, 25.6, and 26.5 degrees two theta±0.2 degrees two-theta, as described in WO 02/43732. The preparation of this crystalline form is exemplified in this PCT publication.

The occurrence of different crystal forms (polymorphism) is a property of some molecules and molecular complexes. A single molecule, like the atorvastatin in formula (I) or the salt complex of formula (II), may give rise to a variety of solids having distinct physical properties like melting point, X-ray diffraction (XRD) pattern, infrared absorption fingerprint, and NMR spectrum. The differences in the physical properties of polymorphs result from the orientation and intermolecular interactions of adjacent molecules (complexes) in the bulk solid. Accordingly, polymorphs are distinct solids sharing the same molecular formula yet having distinct advantageous and/or disadvantageous physical properties compared to other forms in the polymorph family. One of the most important physical properties of pharmaceutical polymorphs is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. On the other hand, where the effectiveness of a drug correlates with peak bloodstream levels of the drug, a property shared by statin drugs, and provided the drug is rapidly absorbed by the GI system, then a more rapidly dissolving form is likely to exhibit increased effectiveness over a comparable amount of a more slowly dissolving form.

The discovery of new polymorphic forms of a pharmaceutically useful compound provides a new opportunity to improve the performance characteristics of a pharmaceutical product. This opportunity is increased even when the obtained polymorphs are of high purity. It enlarges the repertoire of materials that a formulation scientist has available for designing, for example, a pharmaceutical dosage form of a drug with a targeted release profile or other desired characteristic. The importance of pharmaceutical solid polymorphism is described in the Guidance for Industry by the US Department of Health and Humans Services FDA, as well as Polymorphism: in the Pharmaceutical Industry, 2006 WILEY-VCH and Solid-State Chemistry of Drugs by Steohen R. Byrn, Ralph R. Pfeiffer and Joseph G. Stowell (2^{nd} edition, p. 3-5) There is a need in the art for polymorphic forms of atorvastatin hemi-calcium.

### SUMMARY OF THE INVENTION

In one embodiment, the invention provides crystalline atorvastatin hemi-calcium characterized by data selected from a group consisting of: a powder X-ray diffraction (PXRD) pattern having peaks at about 3.2, 7.8, 8.6, 15.5, and 17.7 degrees two theta±0.2 degrees two-theta, and a PXRD pattern substantially as depicted in FIG. 1.

Other embodiments encompass processes for the preparation of the above crystalline atorvastatin hemi-calcium comprising slurrying crystalline atorvastatin hemi-calcium characterized by a PXRD pattern having two peaks at about 5.3 and 8.3 degrees two theta±0.2 degrees two-theta and one broad peak in at 18-23 degrees two theta±0.2 degrees two-theta in tert-butyl-methyl ether (MTBE), and optionally recovering the crystalline atorvastatin hemi-calcium. Preferably, the starting material is in wet form.

In another embodiment, the invention provides crystalline atorvastatin hemi-calcium characterized by data selected from a group consisting of: a PXRD pattern having peaks at about 8.6, 8.9, 10.3, 13.9, and 17.2 degrees two theta±0.2 degrees two-theta, and a PXRD pattern as depicted in FIG. 2.

Other embodiments encompass processes for the preparation of the above crystalline atorvastatin hemi-calcium by recrystallizing atorvastatin hemi-calcium from acetone, ethanol, and water.

Other embodiments encompass pharmaceutical compositions comprising the crystalline atorvastatin hemi-calcium of the present invention and at least one pharmaceutically acceptable excipient.

Other embodiments encompass processes for preparing a pharmaceutical composition, comprising combining the crystalline atorvastatin hemi-calcium of the present invention with a pharmaceutically acceptable excipient.

Other embodiments encompass methods for treating a patient comprising administering a therapeutically effective amount of a pharmaceutical composition comprising the crystalline atorvastatin hemi-calcium of the present invention with a pharmaceutically acceptable excipient, to a patient in need thereof.

### BRIEF DESCRIPTION OF THE FIGURES

FIG. 1 illustrates powder X-ray diffraction pattern for crystalline atorvastatin hemi-calcium characterized by a PXRD pattern having peaks at about 3.2, 7.8, 8.6, 15.5, and 17.7 degrees two theta±0.2 degrees two-theta.

FIG. 2 illustrates powder X-ray diffraction pattern for crystalline atorvastatin hemi-calcium characterized by a PXRD pattern having peaks at about 8.6, 8.9, 10.3, 13.9, and 17.2 degrees two theta±0.2 degrees two-theta.

FIG. 3 illustrates microscopic view of crystalline atorvastatin hemi-calcium of FIG. 1.

FIG. 4 illustrates microscopic view of crystalline atorvastatin hemi-calcium Form I.

FIG. 5 illustrates microscopic view of crystalline atorvastatin hemi-calcium Form II.

FIG. 6 illustrates microscopic view of crystalline atorvastatin hemi-calcium Form III.

FIG. 7 illustrates powder X-ray diffraction pattern for the crystalline atorvastatin Form VIII in US Patent Application Publication No. 2002/0183378.

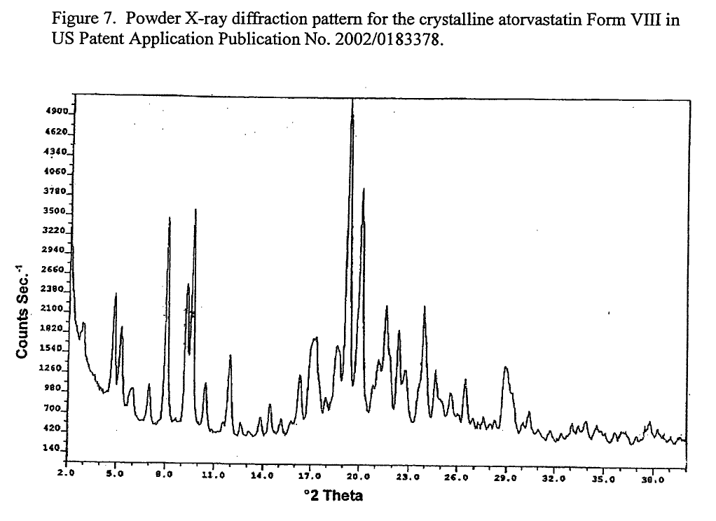

### DETAILED DESCRIPTION OF THE INVENTION

As used herein, the term “room temperature” refers to a temperature of about 15° C. to about 30° C., preferably about 20° C. to about 25° C.

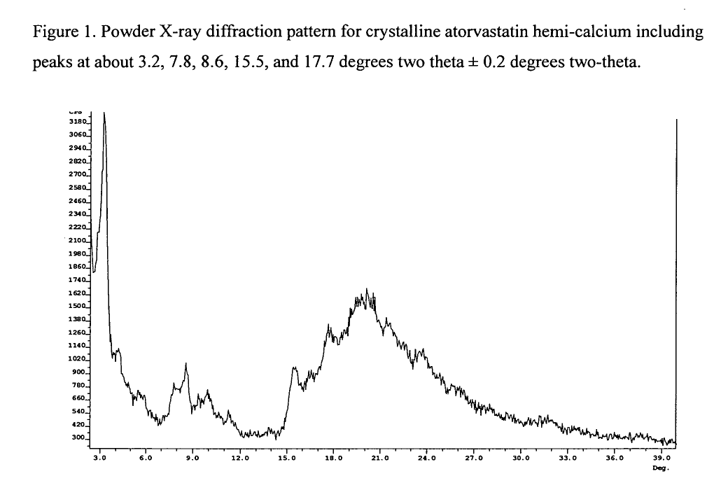

In one embodiment, the invention provides crystalline atorvastatin hemi-calcium, characterized by data selected from a group consisting of: a PXRD pattern having peaks at about 3.2, 7.8, 8.6, 15.5, and 17.7 degrees two theta±0.2 degrees two-theta and a PXRD pattern substantially as depicted in FIG. 1.

The above crystalline atorvastatin hemi-calcium may be further characterized by a PXRD pattern having peaks at about 4.2, 9.3, 10.0, and 11.3, and a broad peak at 18.4-21.2 degrees two theta±0.2 degrees two-theta.

Other embodiments of the invention encompass the above crystalline atorvastatin hemi-calcium containing less than about 50% by weight, preferably, less than 25% by weight, more preferably, less than 10% by weight, even more preferably, less than 5% by weight, most preferably, less than 2% by weight of each one of the crystalline forms of atorvastatin hemi-calcium denominated Form I-IV. Other embodiments of the invention encompass the above crystalline atorvastatin hemi-calcium containing less than about 50% by weight, preferably, less than 25% by weight, more preferably, less than 10% by weight, even more preferably, less than 5% by weight, most preferably, less than 2% by weight of the total weight of the crystalline forms of atorvastatin hemi-calcium denominated Form I-IV.

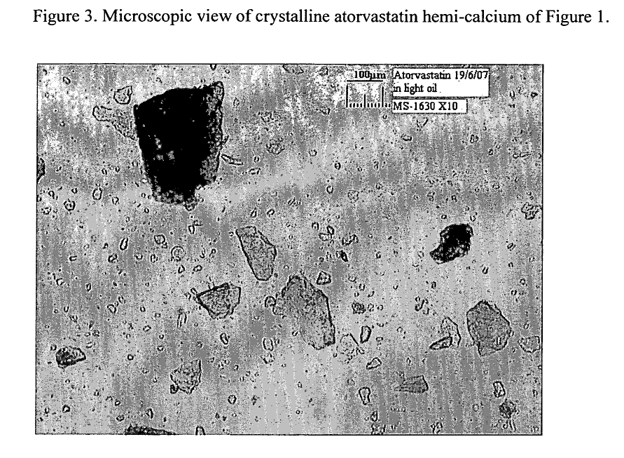

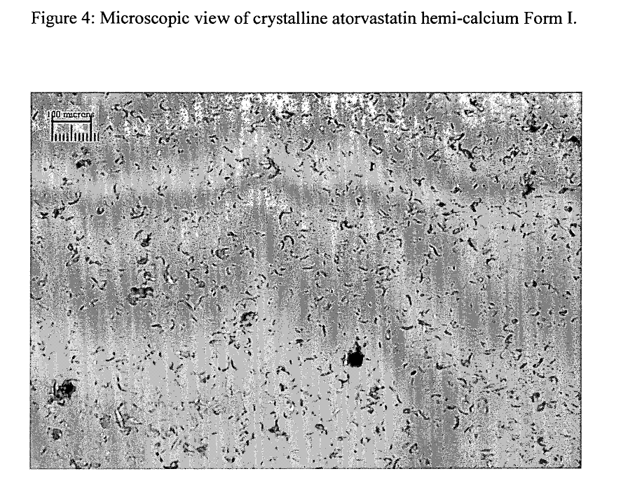

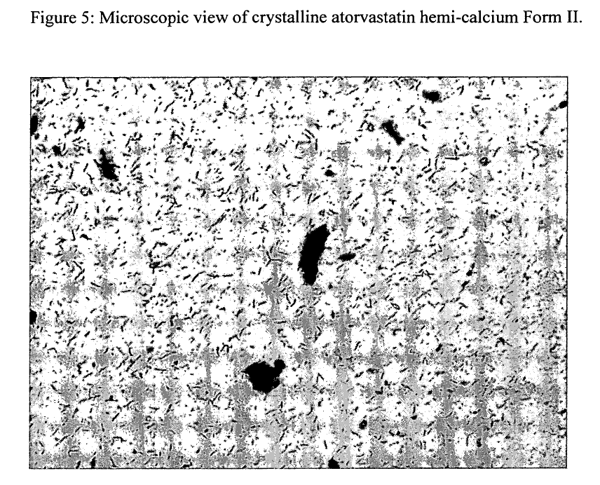

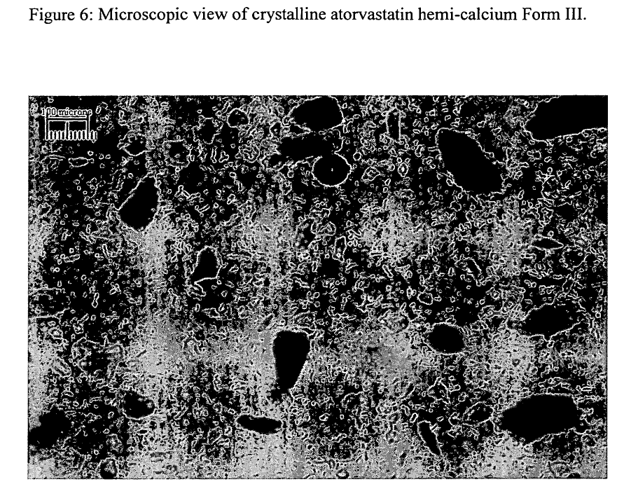

In a preferred embodiment, the above crystalline atorvastatin hemi-calcium is also characterized by an irregular, approximately spherical particle shape, as demonstrated by FIG. 3. Such particle shape is an advantage when comparing it to the needle shape of Forms I-III of the prior art, demonstrated in FIGS. 4-6. Thus, the flowability of crystalline atorvastatin hemi-calcium having such particle shape is improved as compared to the flowability of crystalline atorvastatin hemi-calcium having plate shape or needle shape. The high flowability in the pharmaceutical is an important advantage because several pharmaceutical processes, including blending, transfer, storage, feeding, compaction, and fluidization, involve powder handling. The flow of powder during manufacturing dictates the quality of the product in terms of its weight and content uniformity. Also, the manufacturing efficiency is lower for materials with flowability.

The above crystalline atorvastatin hemi-calcium is prepared by a process comprising slurrying crystalline atorvastatin hemi-calcium characterized by a PXRD pattern having two peaks at about 5.3 and 8.3 degrees two theta±0.2 degrees two-theta and one broad peak in at 18-23 degrees two theta±0.2 degrees two-theta in tert-butyl-methyl ether (MTBE), and optionally recovering the crystalline atorvastatin hemi-calcium. Preferably, the starting material is in wet form.

The starting material for this process can be made by the methods disclosed in the examples of WO01/36384, or by example 3 disclosed herein.

In one embodiment, the slurry is maintained for sufficient time to obtain the crystalline atorvastatin hemi-calcium. Preferably the slurry is maintained for at least 24 hours, preferably about 24 to about 48 hours, more preferably about 26 hours. Preferably the slurry is maintained at room temperature.

The obtained crystalline atorvastatin hemi-calcium may be recovered by any method known in the art, such as filtering out the solvent and/or washing and/or drying the atorvastatin hemi-calcium. Preferably the drying step is at a temperature of from about 40° C. to about 70° C. More preferably, the drying step is at a temperature of about 50° C. to about 65° C., preferably under reduced pressure of less than about 100 mmHg.

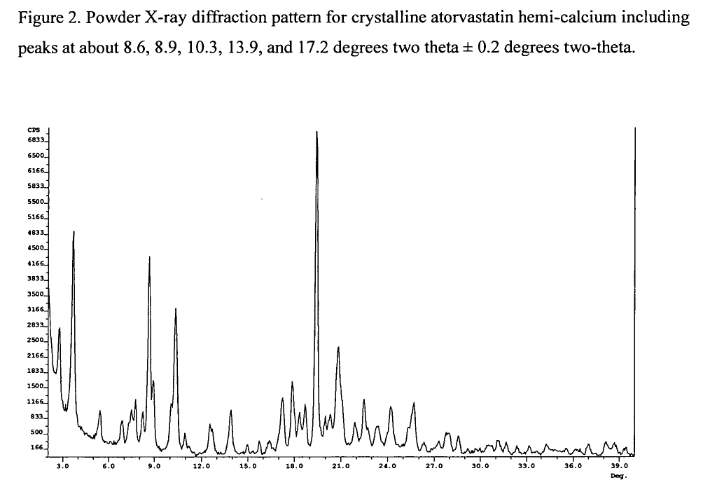

In another embodiment, the invention provides crystalline atorvastatin hemi-calcium, characterized by data selected from a group consisting of: a PXRD pattern having peaks at about 8.6, 8.9, 10.3, 13.9, and 17.2 degrees two theta±0.2 degrees two-theta, and a PXRD pattern as depicted in FIG. 2.

The above crystalline atorvastatin hemi-calcium may be further characterized by a PXRD pattern having peaks at about 3.7, 5.5, 6.9, 7.8, and 17.9 degrees two theta±0.2 degrees two-theta.

Other embodiments of the invention encompass the above crystalline atorvastatin hemi-calcium containing less than about 50% by weight, preferably, less than 25% by weight, more preferably, less than 10% by weight, even more preferably, less than 5% by weight, most preferably, less than 2% by weight of each one of the crystalline forms of atorvastatin hemi-calcium denominated Form I-IV. Other embodiments of the invention encompass the above crystalline atorvastatin hemi-calcium containing less than about 50% by weight, preferably, less than 25% by weight, more preferably, less than 10% by weight, even more preferably, less than 5% by weight, most preferably, less than 2% by weight of the total weight of the crystalline forms of atorvastatin hemi-calcium denominated Form I-IV.

In one embodiment, the invention provides a process for the preparation of the above crystalline atorvastatin hemi-calcium by recrystallizing atorvastatin hemi-calcium from acetone, ethanol, and water. This process also reduces the level of chemical impurities, as will be described below.

The starting material used for the above process may be any crystalline or amorphous form of atorvastatin hemi-calcium, including various solvates and hydrates.

For example, the atorvastatin hemi-calcium starting material may be (1) atorvastatin hemi-calcium characterized by a PXRD pattern having two peaks at about 5.3 and 8.3 degrees two theta±0.2 degrees two-theta and one broad peak at 18-23 degrees two theta±0.2 degrees two-theta, denominated Form V or (2) atorvastatin hemi-calcium characterized by a PXRD pattern having peaks at about 6.9, 9.3, 9.6, 16.3, 17.1, 19.2, 20.0, 21.6, 22.4, 23.9, 24.7, 25.6, and 26.5 degrees two theta±0.2 degrees two-theta, denominated Form VIII.

Form VIII may be prepared by suspending atorvastatin hemi-calcium in a mixture of ethanol and water for a period of time sufficient to convert Form V into Form VIII, substantially as depicted in WO 02/43732.

The starting atorvastatin hemi-calcium is combined with acetone, ethanol, and water to obtain a slurry. The acetone, ethanol, and water can be added separately or as a mixture. The ethanol described herein is preferably absolute ethanol. However, one of ordinary skill in the art could substitute ethanol solutions, such as 95% ethanol, and adjust the amount of water to be combined with the ethanol accordingly.

In one embodiment, the ratio of acetone to the dry weight of atorvastatin hemi-calcium starting material is of about 20 to about 35 ml/g, preferably of about 22 to about 33 ml/g, such as about 28 ml/g.

In one embodiment, the ratio of ethanol to the dry weight of atorvastatin hemi-calcium starting material is about 15 to about 30 ml/g, preferably about 17 to about 27 ml/g, such as about 22 ml/g.

In one embodiment, the ratio of water to the dry weight of atorvastatin hemi-calcium starting material is about 1 to about 10 ml/g, preferably about 2 to about 9 ml/g, such as about 4-7 ml/g. In one embodiment, the ratio is about 6 ml/g.

The atorvastatin hemi-calcium starting material could be dry or wet. When the starting material is wet, the ratios of acetone/ethanol/water to the starting material are calculated based on the dry weight of atorvastatin hemi-calcium in the starting material.

In one embodiment of the invention, the slurry is heated to obtain a solution. Preferably, the heating is to a temperature of from about 50° C. to about 65° C. After dissolution, a gradual precipitation of the crystalline atorvastatin hemi-calcium occurs, providing a suspension. Preferably, the gradual precipitation occurs at a temperature of about 50° C. to about 65° C. The gradual precipitation occurs during a period of about 2.5 to about 24 hours.

The process optionally comprises cooling the suspension to increase yield of the crystalline atorvastatin hemi-calcium. Preferably, the cooling is to a temperature of about room temperature to about 0° C.

Optionally, the cooled suspension can be maintained for a sufficient time to further increase the yield of the crystalline atorvastatin hemi-calcium. Preferably, the cooled suspension is maintained for about 3 to about 5 hours.

The precipitated crystalline atorvastatin hemi-calcium may be recovered by any method known in the art, such as filtering out the solvent and/or washing and/or drying the atorvastatin hemi-calcium. The drying step is preferably at a temperature of from about 40° C. to about 70° C. Preferably, the drying is under reduced pressure.

The recovered crystalline atorvastatin hemi-calcium has a low level of chemical impurities, especially of pyrrole acetonide ester (PAE) of the following formula,

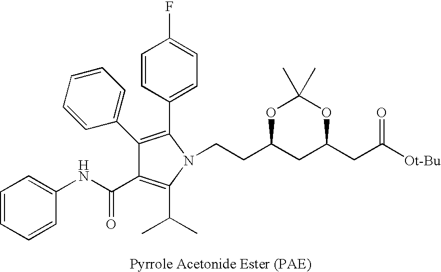

which is the starting material of the synthesis, and of atorvastatin-eliminate (ATV-eliminate) of the following structure,

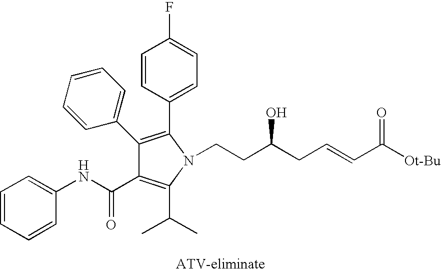

which is an impurity obtained in the last step of the synthesis, and which had previously been difficult to remove from atorvastatin.

The recovered crystalline atorvastatin hemi-calcium contains less than about 0.3% of atorvastatin-eliminate, preferably, less than about 0.1% of atorvastatin-eliminate, more preferably, less than 0.05% of atorvastatin-eliminate. Typically, the levels of chemical impurities are measured by area percent by HPLC.

The invention further provides pharmaceutical formulations comprising the crystalline forms of atorvastatin hemi-calcium of the invention, methods for preparing these formulations, and using them to treat patient in need.

The compositions of the invention include powders, granulates, aggregates, and other solid compositions comprising the solid crystalline forms of atorvastatin hemi-calcium of the invention. In addition, solid formulations that are contemplated by the invention may further include diluents, such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts, and other substituted and unsubstituted celluloses; starch; pregelatinized starch; inorganic diluents, such as calcium carbonate and calcium diphosphate; and other diluents known to the pharmaceutical industry. Other suitable diluents include waxes, sugars, sugar alcohols such as mannitol and sorbitol, acrylate polymers and copolymers, as well as pectin, dextrin, and gelatin.

Further excipients that are within the contemplation of the invention include binders, such as acacia gum, pregelatinized starch, sodium alginate, glucose, and other binders used in wet and dry granulation and direct compression tableting processes. Excipients that also may be present in a solid formulation of the crystalline forms of atorvastatin hemi-calcium of the invention further include disintegrants such as sodium starch glycolate, crospovidone, low-substituted hydroxypropyl cellulose, and others. In addition, excipients may include tableting lubricants such as magnesium and calcium stearate and sodium stearyl fumarate; flavorings; sweeteners; preservatives; pharmaceutically acceptable dyes and glidants such as silicon dioxide.

The dosages include dosages suitable for oral, buccal, rectal, parenteral (including subcutaneous, intramuscular, and intravenous), inhalant, and ophthalmic administration. The most suitable route in any given case will depend on the nature and severity of the condition being treated. In one embodiment of the invention, the route of administration is oral. Dosages may be conveniently presented in unit dosage form and prepared by any of the methods well-known in the art of pharmacy.

Dosage forms include solid dosage forms, such as tablets, powders, capsules, suppositories, sachets, troches, and lozenges, as well as liquid suspensions and elixirs. While the description is not intended to be limiting, the invention is also not intended to pertain to true solutions of atorvastatin hemi-calcium whereupon the properties that distinguish the solid forms of atorvastatin hemi-calcium are lost. However, the use of the novel forms to prepare such solutions is considered to be within the contemplation of the invention.

Capsule dosages contain a solid composition within a capsule, which may be made of gelatin or other conventional encapsulating material. Tablets and powders may be coated with an enteric coating. The enteric-coated powder forms may have coatings comprising phthalic acid cellulose acetate, hydroxypropylmethyl-cellulose phthalate, polyvinyl alcohol phthalate, carboxymethylethylcellulose, a copolymer of styrene and maleic acid, a copolymer of methacrylic acid and methyl methacrylate, and like materials. If desired, suitable plasticizers and/or extending agents may be employed. A coated tablet may have a coating on the surface of the tablet or may be a tablet comprising a powder or granules with an enteric coating.

Having described the invention with reference to certain embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

### Powder X-ray Diffraction

Powder X-ray diffraction data were obtained by using methods known in the art using a SCINTAG powder X-Ray diffractometer model X'TRA equipped with a solid-state detector. Copper radiation of 1.5418 Å was used. A round aluminum sample holder with zero background was used. The scanning parameters included: range: 2-40 degrees two-theta; scan mode: continuous scan; step size: 0.05 deg.; and rate: 5 deg/min. All peak positions are within ±0.2 degrees two theta.

### Determination of Impurity Profile of Atorvastatin Calcium by HPLC

| | |
|---|---|
| Column & Pack- | Synergi Polar RP 80A, 4 μ 250 × 4.6 mm, P/N 00G- |
| ing: | 4336-E0, Phenomenex |
| Buffer: | Mixture of 0.045 M Ammonium Formate and 0.0045 |
| | M Ammonium Acetate. |
| | Adjust pH to 5.0 with 20% Formic acid. |
| Eluent A: | 67% Buffer and 33% Acetonitrile |
| Eluent B: | Acetonitrile |
| Eluent C: | Tetrahydrofuran |

| Gradient | Time | Eluent A, % | Eluent B, % | Eluent C, % |
|---|---|---|---|---|
| | 0 | 91 | 0 | 9 |
| | 15 | 91 | 6 | 3 |
| | 20 | 82 | 16 | 2 |
| | 25 | 82 | 16 | 2 |
| | 50 | 32 | 66 | 2 |
| | 55 | 32 | 66 | 2 |

| | |
|---|---|
| Equilibration | 12 min |
| time: | |
| Sample volume: | 15 μL |
| Flow Rate: | 1.1 mL/min |
| Detector: | 254 nm |
| Column temper- | 40° C. |
| ature: | |
| Diluent | Acetonitrile:Buffer:Tetrahydrofuran 60:35:5 |
| Sample Solution | Prepare 0.5 mg/mL solution of Atrovastatin Calium |
| Preparation | sample in diluent. |
| Qualifying Limit | 0.05% |
| Detection Limit | 0.02% |
| Calculation | $\% impurity i = \frac{Area of impurity i in sample * 100 \%}{Area ATV + Area of impurities}$ |

### Example 1

### Preparation Crystalline Atorvastatin Hemi-Calcium Characterized by Data Selected from a Group Consisting of a PXRD Pattern Having Peaks at About 3.2, 7.8, 8.6, 15.5 and 17.7 Degrees Two Theta±0.2 Degrees Two-Theta

A slurry of atorvastatin hemi-calcium wet Form V (70% by weight of water and ethanol) (10 g) in MTBE (20 ml) was stirred with a mechanical stirrer for 26 hours at room temperature. The product was isolated by a vacuum filtration under nitrogen flow and dried in a vacuum oven at 65° C. for 19.5 hours to obtain 3.4 g of the said crystalline atorvastatin hemi-calcium (84% yield).

### Example 2

### General Procedure for the Preparation of Crystalline Atorvastatin Hemi-Calcium Characterized by Data Selected from a Group Consisting of a PXRD Pattern Having Peaks at About 8.6, 8.9, 10.3, 13.9, and 17.2 Degrees Two Theta±0.2 Degrees Two-Theta

A 1 L reactor was loaded with atorvastatin hemi-calcium wet Form V from Example 3 (30 g) and a mixture of acetone (22-33 ml per gram of dry starting material, which was dried by conventional methods, such as vacuum oven), absolute ethanol (17-27 ml per gram of dry starting material), and water (5.5-9 ml per gram of dry starting material). The slurry obtained was heated to 50° C.-65° C. to obtain complete dissolution. The product precipitated gradually at 50° C.-65° C. during 3-24 hrs. The slurry was then cooled during 1 hour to 0° C. and stirred at 0° C. for 3-5 hrs. The product was isolated by filtration, washing with a mixture of acetone, absolute Ethanol and water at the above ratio (2×50 ml) and drying at 65° C. in a vacuum oven for 8-24 hrs to obtain about 80-90% yield of the said crystalline atorvastatin calcium.

### Example 2a

A 0.5 L reactor was loaded with Atorvastatin hemi-calcium salt Form V wet (10 g, having 54% by weight of water and ethanol) and a mixture of acetone (10 ml per gram of wet starting material), absolute EtOH ethanol (8 ml per gram of wet starting material), and water (2 ml per gram of wet starting material). The slurry obtained was heated to 65° C. for 3 hours. During the heating time the material completely dissolved and then recrystallized from the solution. The slurry was then cooled during 1 hour to 0° C. and stirred at this temperature for 3-5 hours. The product was isolated by filtration, washed with a mixture of acetone, absolute ethanol and water (5:4:1 v/v; 1×6 ml) and dried at 65° C. in a vacuum oven for 24 hours to obtain about 88% yield of the said crystalline atorvastatin hemi-calcium.

Table 1 lists HPLC analysis of atorvastatin hemi-calcium Form V starting material.

TABLE 1

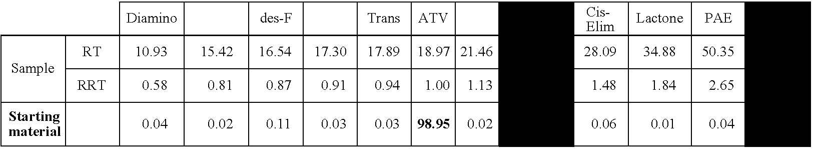

Table 2 lists HPLC analysis of the crystalline product of atorvastatin hemi-calcium.

TABLE 2

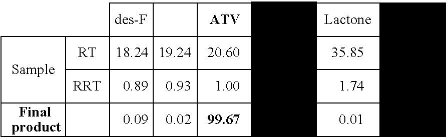

### Example 2b

A 0.5 L reactor was loaded with Atorvastatin hemi-calcium salt Form V wet from production scale (10 g, having 70% by weight of ethanol and water) and a mixture of acetone (10 ml per gram of wet starting material), absolute ethanol (8 ml per gram of wet starting material), and water (2 ml per gram of wet starting material). The slurry obtained was heated to 65° C. for 2.5 hours. During the heating time the material completely dissolved and then recrystallized from the solution. The slurry was then cooled during 1 hour to 0° C. and stirred at this temperature for 3-5 hours. The product was isolated by filtration, washed with a mixture of acetone, absolute Ethanol and water (5:4:1 v/v; 1×6 ml) and dried at 65° C. in a vacuum oven for 24 hours to obtain about 76% yield of the said crystalline atorvastatin hemi-calcium.

Table 3 lists HPLC analysis of atorvastatin hemi-calcium Form V starting material.

TABLE 3

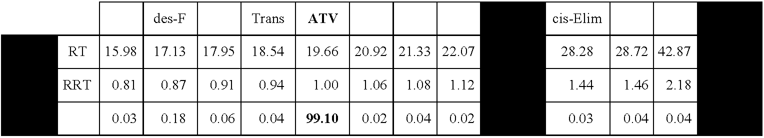

Table 4 lists HPLC analysis of the crystalline product of atorvastatin hemi-calcium.

TABLE 4

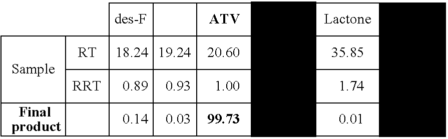

### Example 2c

A 0.5 L reactor was loaded with Atorvastatin hemi-calcium salt Form V wet from production scale (10 g, having 70% by weight of water and ethanol) and a mixture of acetone (10 ml per gram of wet starting material), absolute ethanol (8 ml per gram of wet starting material), and water (2 ml per gram of wet starting material). The slurry obtained was heated to 65° C. for 21 hours. During the heating time the material completely dissolved and then recrystallized from the solution. The slurry was then cooled during 1 hour to 0° C. and stirred at this temperature for 3-5 hours. The product was isolated by filtration, washed with a mixture of acetone, absolute ethanol, and water (5:4:1 v/v; 1×6 ml) and dried at 65° C. in a vacuum oven for 15 hours to obtain about 85% yield of the said crystalline atorvastatin hemi-calcium.

Table 5 lists HPLC analysis of atorvastatin hemi-calcium Form V starting material.

TABLE 5

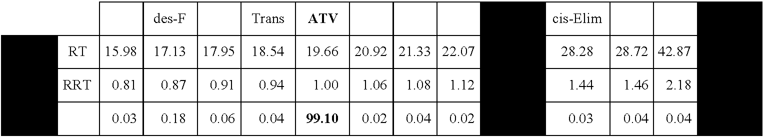

Table 6 lists HPLC analysis of the crystalline product of atorvastatin hemi-calcium.

TABLE 6

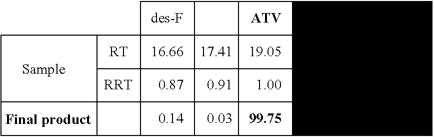

### Example 3

### Preparation of Atorvastatin Hemi-Calcium Wet Form V

Process water (155 kg), 32% HCl (9 kg), absolute ethanol (650 kg), and pyrrole acetonide ester (PAE) (65 kg) were fed into a 2500 L reactor. The reaction mixture was warmed up to about 40° C. and stirred at 79 rpm for 9 hours to obtain a clear solution. Absolute ethanol (260 kg) was added to the reaction mixture, and the additional portion of absolute ethanol (260 kg) was distilled out during 3 hrs at 45° C./61 mmHg. Calcium hydroxide (11.25 kg) was added at 40° C., and the reaction mixture was stirred at 70° C. for 5.5 hrs. The salts was filtrated out and washed with absolute ethanol (37.5 kg). Process water (650 kg) was added at about 64° C. during 34 minutes. The mixture was heated to 82° C., and stirred at this temperature for 15 minutes. The mixture was cooled to 70° C. during 22 minutes, and then to 21° C. during 5 hrs. The obtained slurry was stirred at 21° C. for 3 hrs. The product was filtered by 4 cycles using a centrifuge, and after each cycle was washed with process water (2×18.1 kg). 139.6 kg of wet atorvastatin hemi-calcium salt was obtained, characterized by a PXRD pattern having two peaks at about 5.5 and 7.8 degrees two theta±0.2 degrees two-theta and one broad peak in at 18-23 degrees two theta±0.2 degrees two-theta.

### Example 4

### Preparation of Crystalline Atorvastatin Hemi-Calcium Characterized by Data Selected from a Group Consisting of a PXRD Pattern Having Peaks at About 8.6, 8.9, 10.3, 13.9, and 17.2 Degrees Two Theta±0.2 Degrees Two-Theta

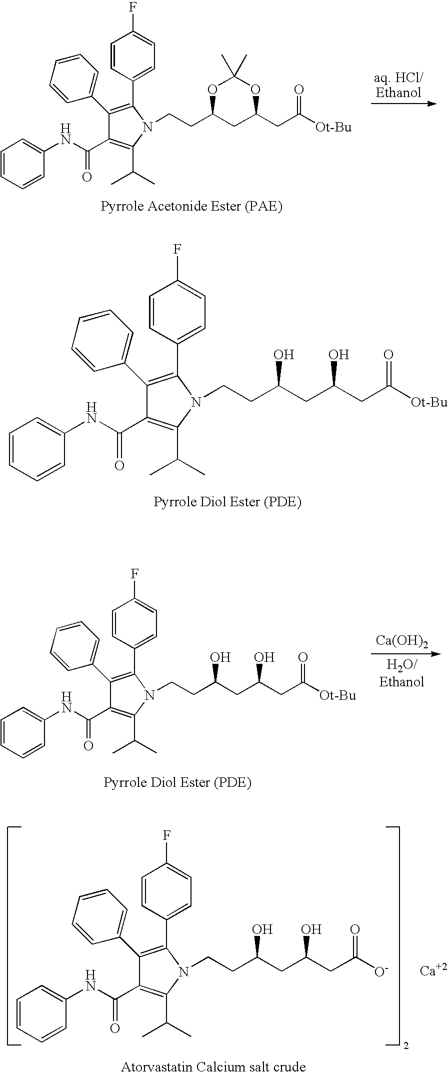

Crude atorvastatin hemi-calcium wet Form V (10 g) from Example 3 was stirred in acetone (10 ml per gram of wet ATV hemi-calcium having 55%-60% of water and ethanol), absolute ethanol (8 ml per gram of wet ATV hemi-calcium), and water (2 ml per gram of wet ATV hemi-calcium) at reflux temperature (65° C.) for 2.5 hrs. During the reflux time, the material dissolved in the mixture of the above solvents. ATV hemi-calcium was then recrystallized from the same mixture. The slurry was then cooled to room temperature and then in an ice-bath. The product was isolated by filtration, washing with a mixture of acetone/absolute ethanol/water at the above ratio (5:4:1 by volume) (1×5 ml), and drying at 65° C. for 24 hrs to obtain the said crystalline ATV hemi-calcium.

Table 7 lists HPLC analysis of atorvastatin hemi-calcium Form V starting material.

TABLE 7

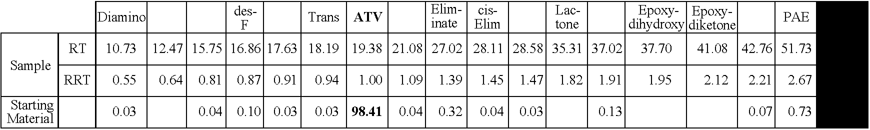

Table 8 lists HPLC analysis of the crystalline product of atorvastatin hemi-calcium.

TABLE 8

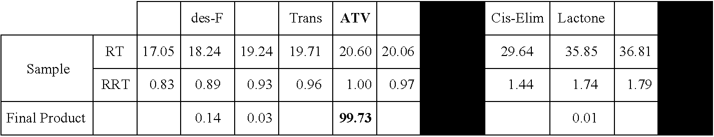

### Example 5

### Preparation of Crystalline Atorvastatin Hemi-Calcium Characterized by Data Selected from a Group Consisting of a PXRD Pattern Having Peaks at About 8.6, 8.9, 10.3, 13.9, and 17.2 Degrees Two Theta±0.2 Degrees Two-Theta

Atorvastatin hemi-calcium dry Form VIII (3 g) was stirred in acetone (22 ml per gram of ATV hemi-calcium dry), absolute ethanol (18 ml per gram of ATV hemi-calcium dry), and water (6 ml per gram of ATV hemi-calcium dry) at reflux temperature (65° C.) for 16 hrs. During the reflux time, the material dissolved in the mixture of the above solvents and recrystallized from the same mixture. The slurry was cooled to room temperature and then in an ice-bath. The product was isolated by filtration, washing with a mixture of acetone/absolute ethanol/water at the above ratio (11:9:3 by volume) (2×5 ml) and drying at 65° C. for 17.5 hrs to obtain the said crystalline ATV hemi-calcium.

Table 9 lists HPLC analysis of the starting atorvastatin hemi-calcium Form VIII and the crystalline product obtained in this example.

TABLE 9

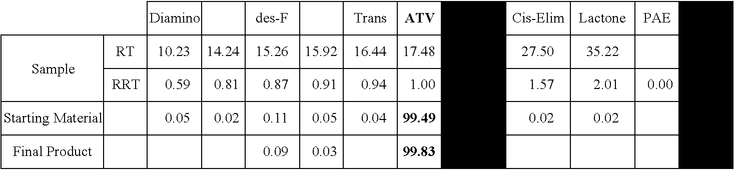

### Table Legend:

Diamino=diamino-atorvastatin

des-F=desfluoro-atorvastatin

Trans=trans-atorvastatin

ATV=Atorvastatin

Eliminate=Atorvastatin eliminate

Cis-Elim=Atorvastatin cis-eliminate

Lactone=Atorvastatin-lactone

1. Crystalline atorvastatin hemi-calcium characterized by data selected from a group consisting of a PXRD pattern with peaks at about 3.2, 7.8, 8.6, 15.5, and 17.7 degrees two theta±0.2 degrees two-theta and a PXRD pattern as depicted in FIG. 1.

2. The crystalline atorvastatin hemi-calcium of claim 1, characterized by a PXRD pattern with peaks at about 3.2, 7.8, 8.6, 15.5, and 17.7 degrees two theta±0.2 degrees two-theta.

3. The crystalline atorvastatin hemi-calcium of claim 1, characterized by a PXRD pattern as depicted in FIG. 1.

4. The crystalline atorvastatin hemi-calcium of claim 1, further characterized by an X-ray powder diffraction pattern with peaks at about 4.2, 9.3, 10.0, 11.3, and a broad peak at 18.4-21.2 degrees two theta±0.2 degrees two-theta.

5. The atorvastatin hemi-calcium of claim 1, containing less than 50% by weight of each of the crystalline atorvastatin hemi-calcium Forms I-IV.

6. The atorvastatin hemi-calcium of claim 5, containing less than 50% by weight of the total weight of crystalline atorvastatin hemi-calcium Forms I-IV.

7. A process for preparing the atorvastatin hemi-calcium of claim 1, comprising slurrying crystalline atorvastatin hemi-calcium characterized by a PXRD pattern having two peaks at about 5.3 and 8.3 degrees two theta±0.2 degrees two-theta and one broad peak in at 18-23 degrees two theta±0.2 degrees two-theta in tert-butyl-methyl ether (MTBE).

8. A process according to claim 7, further comprising the step of:
recovering the crystalline atorvastatin hemi-calcium.

9. Crystalline atorvastatin hemi-calcium characterized by data selected from a group consisting of a PXRD pattern with peaks at about 8.6, 8.9, 10.3, 13.9, and 17.2 degrees two theta±0.2 degrees two-theta and a PXRD pattern as depicted in FIG. 2.

10. The crystalline atorvastatin hemi-calcium of claim 9, characterized by a PXRD pattern with peaks at about 8.6, 8.9, 10.3, 13.9, and 17.2 degrees two theta±0.2 degrees two-theta.

11. The crystalline atorvastatin hemi-calcium of claim 9, characterized by a PXRD pattern as depicted in FIG. 2.

12. The crystalline atorvastatin hemi-calcium of claim 9, further characterized by an X-ray powder diffraction pattern with peaks at about 3.7, 5.5, 6.9, 7.8, and 17.9 degrees two theta±0.2 degrees two-theta.

13. The crystalline atorvastatin hemi-calcium of claim 9, containing less than 50% of each of the crystalline atorvastatin hemi-calcium Forms I-IV.

14. The atorvastatin hemi-calcium of claim 13, containing less than 50% by weight of the total weight of crystalline atorvastatin hemi-calcium Forms I-IV.

15. A process for preparing the crystalline atorvastatin hemi-calcium as defined in claim 9, comprising the steps of:
recrystallizing atorvastatin hemi-calcium from acetone, ethanol, and water.

16. A process of claim 15, wherein the starting atorvastatin hemi-calcium is combined with acetone, ethanol, and water to obtain a slurry.

17. A process according to claim 15, wherein the starting atorvastatin hemi-calcium is selected from
(i) atorvastatin hemi-calcium characterized by a PXRD pattern having two peaks at about 5.3 and 8.3 degrees two theta±0.2 degrees two-theta and one broad peak at 18-23 degrees two theta±0.2 degrees two-theta, and
(ii) atorvastatin hemi-calcium characterized by a PXRD pattern having two sharp peaks at about 9.3 and 9.6 degrees two theta±0.2 degrees two-theta.

18. A process according to an claim 15, wherein the ratio of acetone to the dry weight of atorvastatin hemi-calcium starting material is of about 20 to about 35 ml/g.

19. A process according to claim 15, wherein the ratio of ethanol to the dry weight of atorvastatin hemi-calcium starting material is about 15 to about 30 ml/g.

20. A process according to claim 15, wherein the ratio of water to the dry weight of atorvastatin hemi-calcium starting material is about 1 to about 10 ml/g.

21. A process according to claim 15, wherein the slurry is heated to a temperature of from about 50° C. to about 65° C. to obtain a solution.

22. A process according to claim 15, wherein atorvastatin hemi-calcium is dissolved, and wherein after dissolution, a gradual precipitation of the crystalline atorvastatin hemi-calcium occurs, providing a suspension.

23. A process according to claim 22, wherein the gradual precipitation occurs at a temperature of about 50° C. to about 65° C.

24. A process according to claim 22, further comprising cooling the suspension to a temperature of about room temperature to about 0° C.

25. A process according to claim 15, further comprising the step of: recovering the crystalline atorvastatin hemi-calcium.

26. A pharmaceutical composition comprising the crystalline atorvastatin hemi-calcium of claim 1, and at least one pharmaceutically acceptable excipient.

27. A process for preparing the pharmaceutical composition of claim 26, comprising combining the crystalline atorvastatin hemi-calcium with the pharmaceutically acceptable excipient.

28. (canceled)

29. A method of treatment of hypercholesterolaemia or a method for reducing the risk of cardiovascular events in diabetic patients, comprising administering the crystalline atorvastatin hemi-calcium of claim 1.

30. A pharmaceutical composition comprising the crystalline atorvastatin hemi-calcium of claim 9 and at least one pharmaceutically acceptable excipient.

31. A method of treatment of hypercholesterolaemia or a method for reducing the risk of cardiovascular events in diabetic patients, comprising administering the crystalline atorvastatin hemi-calcium of claim 9.
